(19) United States
(12) Patent Application Publication
(10) Pub. No.: US 2022/0362000 A1
(43) Pub. Date: Nov. 17, 2022

(54) Apparatus for Treating Urinary Incontinence, and Panties Having Same

(71) Applicant: RIM Healthcare Co., Ltd., Seoul (KR)

(72) Inventor: Khae Hwan Kim, US (US)

(21) Appl. No.: 17/771,456

(22) PCT Filed: Oct. 27, 2020
(86) PCT No.: PCT/KR2020/014707

§ 371 Date: Apr. 22, 2022

(30) Foreign Application Priority Data
Nov. 1, 2019 (KR) 10-2019-0138860

(51) Int. Cl. A61F 2/00; A41B 9/04

(52) U.S. Cl. CPC A61F 2/005; A61F 2/0009; A41B 9/04; A41B 2400/32

(57) ABSTRACT

Proposed is an apparatus for treating urinary incontinence. The apparatus for treating urinary incontinence includes a locking part having one side in contact with the external genitalia of a woman, an insertion part protruding from the one side of the locking part and extending a preset length so as to be inserted into the vagina of the woman; a contact part protruding forward from the insertion part so as to contact a specific point, on the front side of the vaginal wall, corresponding to a connection part between the distal urethra and the central urethra, and a support part protruding rearward in the opposite direction from the contact part so as to contact the rear side of the vaginal wall. The support part protrudes longer than the contact part to maintain contact with the specific point of the contact part.

### CROSS-REFERENCE TO RELATED APPLICATIONS

This non-provisional patent application is a national phase entry filed under 35 U.S.C. § 371 of International Patent Application No. PCT/KR2020/014707, filed on Oct. 27, 2020, the contents of which are hereby incorporated by reference in their entirety.

### BACKGROUND OF THE INVENTION

### Field of the Invention

The present disclosure relates to an apparatus for treating urinary incontinence. More specifically, the present disclosure relates to an apparatus for treating urinary incontinence, the apparatus being capable of preventing incontinence by being inserted into a women's vagina, and relates to underpants having the apparatus.

### Description of Related Art

Urinary incontinence is a disease in which urine is discharged regardless of a person's will, and frequently occurs in middle-aged women. Such urinary incontinence may be divided into stress urinary incontinence in which urine is discharged without contraction of the bladder when an abdominal pressure suddenly increases, such as coughing, sneezing, skipping with a jump rope, and so on, and urge urinary incontinence in which a large amount of urine is discharged out of control while feeling an urge to urinate, and so on. As a method of treating such urinary incontinence, there are a surgical treatment such as raising a bladder neck or reducing a urethra and a non-surgical treatment represented by Kegel exercise.

In Patent Document 1 (Korean Patent No. 10-1130273), there is provided a sport apparatus for a sphincter, the sport apparatus being inserted into a woman's vagina to induce Kegel exercises such that a muscle at an entrance side of the vagina is strengthened. In the patent document described above, it has been described that urinary incontinence can be prevented since a body part inserted into the woman's vagina increases a contact area of a portion pressing a urethra portion. However, when an object inserted into the vagina presses the entire area toward the urethra, the object may irritate a portion inducing incontinence, so that the object does not prevent incontinence but induces incontinence.

By research results of the present inventor, it has been confirmed that incontinence is induced when a connection region between the proximal urethra and the bladder. Further, In the Patent Document described above, a convex portion positioned on an upper portion among two convex portions indicated by reference numeral 23 in FIG. 6 irritates the connection region between the proximal urethra and the bladder, and it is believed that the convex portion will eventually induce incontinence.

In addition, according to the Patent Document described above, a locking member indicated by reference numeral 10 is formed to surround a wearer's anterior pubic portion, and is in contact with the urethral opening and the clitoris. When the urethral opening and the clitoris is irritated by a contact, incontinence is not prevented but is induced, and the wearer feels foreign body sensation. Further, since the sport apparatus for the sphincter disclosed in Patent Document described above is for performing Kegel exercises and has a structure that fully fills the vagina, the wearer's feeling of the foreign body sensation in the vagina cannot be avoided and the sport apparatus for the sphincter is not suitable for an apparatus that is required to be worn for a long time to prevent urinary incontinence.

### BRIEF SUMMARY OF THE INVENTION

### Disclosure Technical Problem

Accordingly, the present disclosure has been made keeping in mind the above problems occurring in the related art, and an objective of the present disclosure is to provide an apparatus for treating urinary incontinence, the apparatus being inserted inside a woman's vagina and being capable of preventing an incontinence situation that occurs regardless of the woman's intention just by being in contact with a specific region inside the vagina.

In addition, another objective of the present disclosure is to provide an apparatus for treating urinary incontinence which is capable of preventing the incontinence situation as described above and which is capable of minimizing a foreign body sensation that may occur when the apparatus is inserted into the woman's vagina.

In addition, still another objective of the present disclosure is to provide an apparatus for treating urinary incontinence, the apparatus being provided on underpants and being capable of being in contact with an intended specific position by adjusting the underpants via an outerwear, thereby being capable of increasing a wearer's convenience.

### Technical Solution

According to an embodiment of the present disclosure, there is provided an apparatus for treating urinary incontinence, the apparatus including: a locking part provided with a first surface that is in contact with a genital skin of a woman; an insertion part which protrudes from the first surface of the locking part and which extends to a predetermined length such that the insertion part is inserted into the woman's vagina; a contact part that protrudes forward from the insertion part such that the contact part is in contact with a first point on a front side of the woman's vaginal wall, the first point corresponding to a connection part between the woman's distal urethra and the woman's central urethra; and a support part that protrudes rearward in an opposite direction from the contact part such that the support part is in contact with a rear side of the woman's vaginal wall. Here, the support part may protrude longer than the contact part such that a state in which the contact part is in contact with the first point is maintained.

According to an embodiment of the present disclosure, the insertion part may be configured such that a cross-sectional area thereof is gradually decreased and then increased from at a part where the contact part or the support part is formed to the locking part.

According to an embodiment of the present disclosure, the contact part may protrude by at least 2 mm but less than or equal to 4 mm from a foremost point that is spaced apart by a predetermined distance from the first surface of the locking part in a length direction of the insertion part.

According to an embodiment of the present disclosure, the support part may protrude by at least 5 mm but less than or equal to 7 mm from a rearmost point that is spaced apart by the predetermined distance from the first surface of the locking part in the length direction of the insertion part.

According to an embodiment of the present disclosure, the predetermined distance may be at least 10 mm but less than or equal to 15 mm.

According to an embodiment of the present disclosure, a foremost protruding point of the contact part may be positioned closer to the first surface of the locking part than a rearmost protruding point of the support part.

According to an embodiment of the present disclosure, the foremost protruding point may be spaced apart from the first surface of the locking part by at least 20 mm and less than or equal to 25 mm.

According to an embodiment of the present disclosure, an end portion surface of the insertion part may be upwardly inclined with respect to a front-to-back direction.

According to an embodiment of the present disclosure, a preset length of the insertion part may be less than or equal to 35 mm.

According to an embodiment of the present disclosure, the locking part may include an anterior extension part that extends forward and a posterior extension part that extends rearward, and the posterior extension part may extend longer than the anterior extension part.

According to an embodiment of the present disclosure, the anterior extension part may extend such that an end portion of the anterior extension portion at the front side is positioned between the woman's urethra and the woman's vagina in order for the anterior extension part to be not in contact with the woman's urethra when the insertion part is inserted into the woman's vagina.

According to an embodiment of the present disclosure, a width of the posterior extension part in a left and right direction may be gradually decreased toward a rear side of the posterior extension part.

According to an embodiment of the present disclosure, there is provided underpants. The underpants may include: the apparatus for treating urinary incontinence; a wearing part which forms an external shape of the underpants and which is formed to be worn on a woman's body; and a coupling part formed such that the apparatus for treating urinary incontinence is mounted.

According to an embodiment of the present disclosure, the coupling part may be formed of at least two layers of cloths, and the locking part may be coupled to the underpants by being fitted between the cloth layers.

In addition, an additional configuration may be further included in the apparatus for treating urinary incontinence according to an embodiment of the present disclosure and or in the underpants including the apparatus.

### Advantageous Effects

According to an embodiment of the present disclosure, when the apparatus is inserted into the woman's vagina and is brought into contact with the specific position inside the vagina, the incontinence situation that occurs regardless of the woman's intention may be prevented.

According to an embodiment of the present disclosure, since the support part extends longer than the contact part, a state in which the contact part is in contact with the first point may be maintained even if the wearer walks freely while wearing the apparatus.

According to an embodiment of the present disclosure, since the insertion part inserted into the woman's vagina is formed in the preset length such that the contact of the apparatus with a portion other than the specific region is minimized, the wearer's foreign body sensation may be minimized.

According to an embodiment of the present disclosure, since the locking part extends rearward, the locking part stably supports the insertion part. Therefore, since the locking part helps the contact part to maintain the contact state and also the anterior extension part of the locking part does not contact the urethra, incontinence caused by irritating the urethra may be prevented.

According to an embodiment of the present disclosure, since the apparatus for treating urinary incontinence is provided on the underpants, the underpants can be adjusted via the outerwear. Accordingly, even if the apparatus for treating urinary incontinence is not in contact with the specific position, a position of the apparatus for treating urinary incontinence can be easily adjusted to a correct position by adjusting the underpants via the outerwear. Therefore, the wearer's convenience may be increased.

### BRIEF DESCRIPTION OF THE SEVERAL VIEWS OF THE DRAWINGS

FIG. 1 is a photo showing a position between the central urethra and the distal urethra, the position being a contact part of an apparatus for treating urinary incontinence according to an embodiment of the present disclosure is in contact with;

FIGS. 2A and 2B are photos showing a principle of the apparatus for treating urinary incontinence according to an embodiment of the present disclosure;

FIG. 3 is a perspective view illustrating the apparatus for treating urinary incontinence according to an embodiment of the present disclosure;

FIG. 4 is a side view illustrating the apparatus for treating urinary incontinence according to an embodiment of the present disclosure;

FIG. 5 is a view schematically illustrating a state in which the apparatus for treating urinary incontinence according to an embodiment of the present disclosure is worn;

FIG. 6 is a view schematically illustrating a woman's genital area so as to describe a contact region of the apparatus for treating urinary incontinence according to an embodiment of the present disclosure;

FIG. 7 is a plan view illustrating the apparatus for treating urinary incontinence according to an embodiment of the present disclosure; and

FIG. 8 is a view schematically illustrating underpants provided with the apparatus for treating urinary incontinence according to an embodiment of the present disclosure.

### DETAILED DESCRIPTION OF THE INVENTION

Best Mode

Hereinafter, preferred embodiments of the present disclosure will be described in detail with reference to the appended drawings to such an extent that the present disclosure can be readily practiced by one of ordinary skill in the art.

Detailed descriptions of parts irrelevant to the present disclosure will be omitted for the purpose of more clearly describing the present disclosure. Throughout the specification, the same or similar components will be described using same or similar reference numerals. In addition, the sizes, thicknesses, positions, and the like of the respective components shown in the drawings are arbitrarily illustrated for the sake of convenience of explanation, and hence the present disclosure is not necessarily limited thereto. That is, it should be understood that specific shapes, configurations, and characteristics described in the specification may be modified in various embodiments without departing from the spirit and scope of the prevent disclosure, and positions or arrangements of individual components may be modified without departing from the spirit and scope of the prevent disclosure.

Therefore, detailed descriptions to be described below should be construed as non-limitative senses, and the scope of the prevent disclosure should be understood to include the scope of the appended claims and all equivalents thereof.

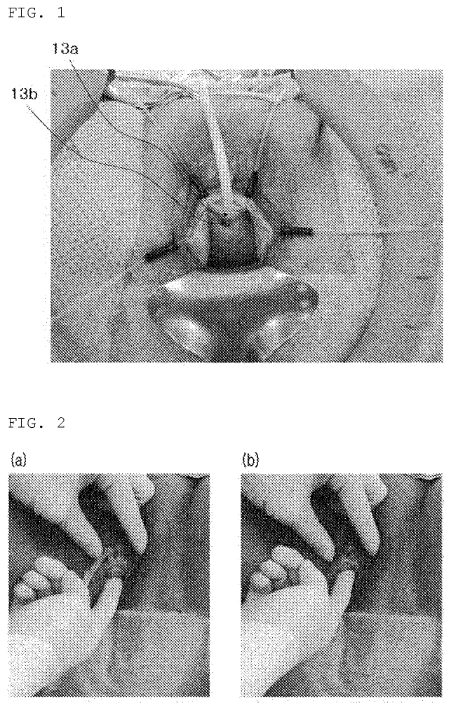

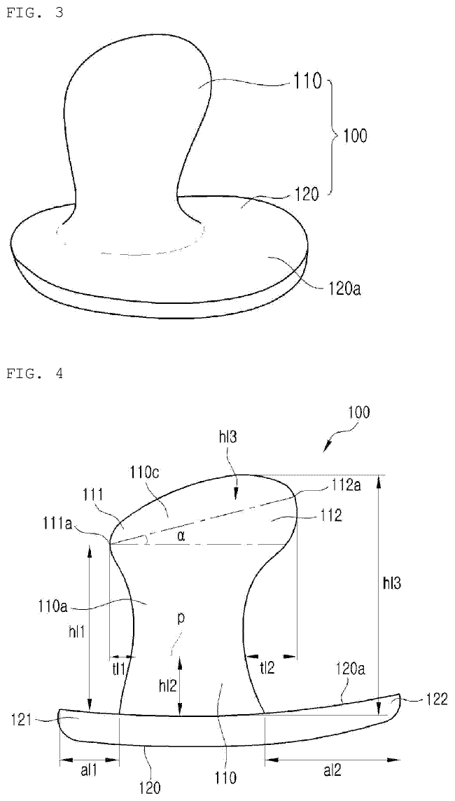

FIG. 1 is a photo showing a position between a central urethra and a distal urethra, the position being a contact part of an apparatus for treating urinary incontinence according to an embodiment of the present disclosure is in contact with. FIGS. 2A and 2B are photos showing a principle of the apparatus for treating urinary incontinence according to an embodiment of the present disclosure.

The present inventor(s) has found that incontinence can be stopped by touching or lightly pressing a specific point in a woman's vaginal wall, the specific point corresponding to a position between a distal urethra and a central urethra.

Referring to FIG. 1, a pudenda of an incontinence patient who is positioned in a litomy position is seen. A Foley catheter is inserted into the incontinence patient's urethra, and a hole is formed between the urethra and a vaginal wall due to previous urinary incontinence surgery. A part where the Foley catheter is seen through the hole is the specific point (hereinafter, referred to as a first point 13b) of an anterior vaginal wall, the specific point stopping the incontinence.

Generally, a woman's urethral canal has a length of about 4 cm to 45 cm, from a woman's urethral opening (Female urethra) to a woman's urinary bladder. The urethral canal is divided into a distal urethra 12a, a central urethra 12b, and a proximal urethra 12c. Further, the first point 13b corresponds to a part connecting the distal urethra 12a and the central urethra 12b. The first point 13b is positioned at the vaginal wall corresponding to a position about 20 mm to 25 mm into a body from the urethral opening. That is, the first point 13b is positioned at the anterior vaginal wall at a position in a proximal direction about 20 mm to 25 mm from an entrance of a woman's vagina 13.

Referring to FIGS. 2A and 2B, FIG. 2A is a photo showing a state before an index finger is in contact with the first point 13b, and urine is discharged through the urethra regardless of the patient's will due to urge urinary incontinence or stress urinary incontinence. FIG. 2B is a photo showing a state in which a portion of a knuckle of the index finger is in contact with the first point 13b, and urine being discharged regardless of the patient's will is stopped as illustrated in FIG. 2B.

In FIG. 2B, when a portion of a first knuckle of the index finger is in a state of being in contact with the first point 13b, an end portion of the index finger is in contact with and supports a posterior vaginal wall of the vagina 13. Here, a front direction may refer to an anterior direction where a person's face or belly is facing. In other words, a direction facing the front direction may refer to a posteroanterior direction. A rear direction refers to a direction opposite to the front direction, and a direction facing the rear direction may refer to an anteroposterior direction.

An apparatus for treating urinary incontinence according to an embodiment of the present disclosure is proposed by conceiving a shape of the index finger so as to prevent urinary incontinence in daily life, the index finger being in contact with the first point 13b when a woman is in a state in which urinary incontinence is stopped.

Hereinafter, the apparatus for treating urinary incontinence according to an embodiment of the present disclosure will be described in detail with reference to the drawings.

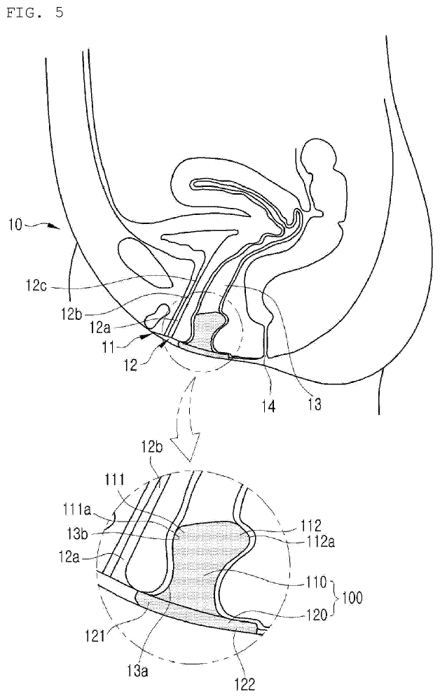

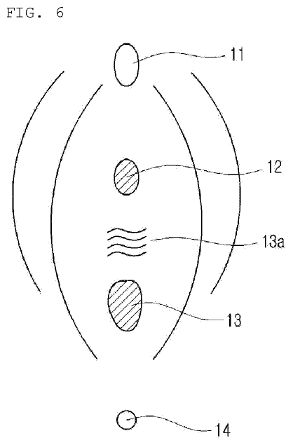

FIG. 3 is a perspective view illustrating the apparatus for treating urinary incontinence according to an embodiment of the present disclosure, FIG. 4 is a side view illustrating the apparatus for treating urinary incontinence according to an embodiment of the present disclosure, and FIG. 5 is a view schematically illustrating a state in which the apparatus for treating urinary incontinence according to an embodiment of the present disclosure is worn.

The apparatus 100 for treating urinary incontinence according to an embodiment of the present disclosure includes an insertion part 110 and a locking part 120. The insertion part 110 is configured to prevent incontinence by being inserted into the woman's vagina 13 and being in contact with the first point 13b of the vaginal wall, the first point 13b corresponding to the portion connecting the distal urethra 12a and the central urethra 12b. Further, the locking part 120 is configured such that the genital skin of the woman is caught on the locking part 120 so that the insertion part 110 is prevented from entering deep into the woman's vagina 13.

According to an embodiment of the present disclosure, the insertion part 110 may have a generally cylindrical shape, and an outer circumferential surface of the insertion part 110 may be formed in a geometrical shape having a continuous flexure. In addition, the insertion part 110 may extend in a first direction such that the insertion part 110 has a predetermined length as described later.

The locking part 120 may be formed in an oval shape when viewed in a plan view. The locking part 120 includes a first surface 120a that is in contact with the genital skin of the woman. The first surface 120a may be formed in a curved shape that is concaved toward a center of the first surface 120a.

According to an embodiment of the present disclosure, the insertion part 110 includes a contact part 111 and a support part 112. On the insertion part 110, the contact part 111 protrudes toward the anterior direction. More specifically, the contact part 111 may protrude toward the anterior direction from a first portion on the outer circumferential surface of the insertion part 110 directed toward the anterior direction. When the insertion part 110 is in a state of being inserted into the woman's vagina 13, the contact part 111 may be in contact with the first point 13b of the vaginal wall, the first point 13b corresponding to the connecting part between the distal urethra 12a and the central urethra 12b.

The support part 112 protrudes toward the posterior direction that is opposite to the anterior direction. More specifically, the support part 112 may protrude toward the posterior direction from a portion opposite to the first portion of the outer circumferential surface of the insertion part 110 on which the contact part 111 is formed. The support part 112 is in contact with a posterior side of the vaginal wall (the posterior vaginal wall), so that the support part 112 may support the contact part 111 such that the contact part 111 maintains a state of being in contact with the first point 13b.

Since the first point 13b is positioned 20 mm to 25 mm into the body from the entrance of the vagina 13, the contact part 111 may be formed at a point corresponding to a first height hl1 of at least 20 mm but less than or equal to 25 mm in a length direction of the insertion part 110 on the first surface 120a of the locking part 120. Here, the first height hl1 may refer to a height to a foremost protruding point 111a from a point of the first surface 120a that is spaced apart in the length direction of the insertion part 110 from the foremost protruding point 111a of the contact part 111. However, when the contact part 111 and the support part 112 excessively protrude on the outer circumferential surface of the insertion part 110 and excessively press the first point 13b, incontinence may be induced, and foreign body sensation may be induced since the vaginal wall is irritated. Therefore, a protruding length of the contact part 111 may be limited to have a predetermined length.

Since the outer circumferential surface of the insertion part 110 is formed in the curved shape corresponding to an inside of the vaginal wall that is curved, the protruding lengths of the contact part 111 and the support part 112 are required to be determined on the basis of a certain reference point. Hereinafter, the outer circumferential surface 110a of the insertion part 110 and the protruding lengths of the contact part 111 and the support part 112 will be described in detail.

According to an embodiment of the present disclosure, a center portion of the insertion part 110 in the length direction of the insertion part 110 may be concaved such that the insertion part 110 corresponds to a shape of an inner circumferential surface of the vaginal wall at the entrance side of the woman's vagina. That is, the insertion part 110 may be formed such that an area of a cross-section of the insertion part 110 is gradually decreased and then increased up to the locking part 120 from a portion where the contact part 111 or the support part 112 is formed.

In the shape of the insertion part 110 that is formed as described above, each of the contact part 111 and the support part 112 may protrude to respectively have a first protruding length tl1 and a second protruding length tl2 on the basis of the reference point p. Hereinafter, the reference point p will be described in detail with reference to the drawings.

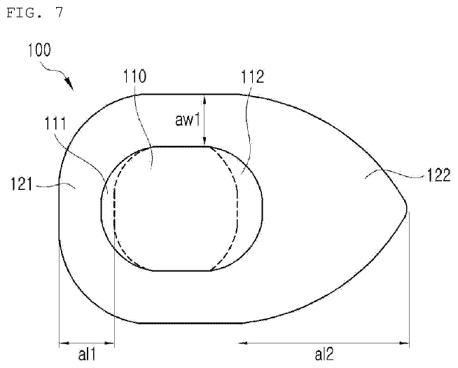

FIG. 6 is a view schematically illustrating a woman's genital area so as to describe a contact region of the apparatus for treating urinary incontinence according to an embodiment of the present disclosure.

In FIG. 6, a clitoris 11, the urethra 12, the vagina 13, and an anus 14 of the woman are illustrated. Generally, creases called Rugae are formed at a region 13a between the urethra 12 and the vagina 13. However, when a man's penis is inserted into a woman's vagina, the region 13a where the Rugae is formed is pushed into the vagina 13, and friction with the man's penis occurs. Therefore, in middle-aged women, the Rugae at the region 13a is disappeared, and becomes a flat state or a smooth state.

In the same manner, when the apparatus 100 for treating urinary incontinence according to an embodiment of the present disclosure is inserted into the woman's vagina 13, the region 13a where the Rugae is formed pushed up. Even if contact or pressure is applied on the region 13a that is the flat state or the smooth state due to the absence of the Rugae, the region 13a has no effect on preventing or causing urinary incontinence. Therefore, a portion that is in contact with the region 13a may be used as the reference point p of the first protruding length tl1 and the second protruding length tl2.

According to an embodiment of the present disclosure, the reference point p is a point corresponding to a second height hl2 from the center of the insertion part 110 on the first surface 120a of the locking part 120 toward the length direction of the insertion part 110. Here, the second height hl2 may be at least 10 mm but less than or equal to 15 mm.

Meanwhile, the contact part 111 may protrude from a foremost portion of the outer circumferential surface 110a such that the contact part 111 has the first protruding length tl1 from the reference point p. Here, the first protruding length tl1 may be at least 2 mm but less than or equal to 4 mm, and may preferably be about 3 mm. In addition, the support part 112 may protrude from a rearmost portion of the outer circumferential surface 110a such that the support part 112 has the second protruding length tl2 from the reference point p. Here, the second protruding length tl2 may be at least 5 mm but less than or equal to 7 mm, and may preferably be about 6 mm.

As the contact part 111 and the support part 112 that are described above protrude to the first protruding length tl1 and the second protruding length tl2 from the reference point p, respectively, in a state in which the apparatus 100 for treating urinary incontinence is inserted into the vagina 13, the apparatus 100 for treating urinary incontinence may touch or lightly press the first point 13b without excessive pressure, thereby preventing incontinence that occurs regardless of the patient's will.

Meanwhile, in middle-aged women with urinary incontinence, bladder ptosis, also called as cystocele or prolapsed bladder, occurs, and the anterior vaginal wall of the distal urethra 12a and the central urethra 12b is moved in an anterior and inferior direction. Here, since the posterior vaginal wall facing the first point 13b is fixed relatively, the first point 13b of the anterior vaginal wall and a corresponding region of the posterior vaginal wall are positioned obliquely from each other along the proximal direction. That is, the first point 13b is positioned closer to the entrance of the vagina 13 than the corresponding region of the posterior vaginal wall.

In the vagina 13 in this state, in order to minimize resistance and to minimize the foreign body sensation during an insertion period, the contact part 111 may be formed to be positioned below the support part 112 when the apparatus 100 for treating urinary incontinence is inserted into the vagina 13. That is, the foremost protruding point 111a and a rearmost protruding point 112a is required to be positioned obliquely from each other along the length direction of the insertion portion 110 such that the foremost protruding point 111a and the rearmost protruding point 112b respectively correspond to the first point 13b and the corresponding region of the posterior vaginal wall.

In other words, from the foremost protruding point 111a to the rearmost protruding point 112a, the contact part 111 and the support part 112 may be inclined upwardly. At this time, a relationship between the foremost protruding point 111a and the rearmost protruding point 112a can be explained by an inclination angle as follows.

The foremost protruding point 111a and the rearmost protruding point 112a may be formed to have a predetermined inclination angle α that is an angle between an imaginary line connecting a space between the foremost protruding point 111a and the rearmost protruding point 112a and another imaginary line which intersects perpendicularly to the length direction of the insertion part 110. Further, the inclination angle α may be nine degrees to twenty degrees.

Here, the inclination angle α of nine degrees to ten degrees is a value in consideration of a general case of urinary incontinence patients who have a cystocele phenomenon, and the inclination angle α of fifteen degrees to twenty degrees is a value in consideration of a serious case in which a urethral prolapse phenomenon occurs.

To explain again by using a relationship between the first surface 120a and both the contact part 111 and the support part 112, the support part 112 may be formed at a position farther from the first surface 120a of the locking part 120 than the contact part 111. In other words, the foremost protruding point 111a of the contact part 111 may be positioned closer to the first surface 120a of the locking part 120 than the rearmost protruding point 112a of the support part 112. The position of the support part 112 as described above may be substantially the same as the position of the end portion of the index finger when a first knuckle of the index finger is brought into contact with the first point 13b as described above.

Meanwhile, when the entire urethral canal 12a, 12b, and 12c is contacted or pressed, incontinence may be induced. Specifically, when a connecting part between the proximal urethra 12c and the bladder is pressed, urge urinary incontinence is induced, so that incontinence may occur before the woman takes down an underwear. Accordingly, in order to prevent a region other than the first point 13b from being irritated, the insertion part 110 may be formed to have a predetermined length hl3.

Here, the predetermined length hl3 may be less than or equal to 35 mm. However, since the contact part 111 is formed to be spaced apart by 25 mm from the first surface 120a of the locking part 120, the predetermined length hl3 may be at least 25 mm but less than or equal to 35 mm. In addition, the predetermined length hl3 may refer to a length between a point farthest from the first surface 120a of the locking part 120 and a point on the first surface 120a spaced apart toward the length direction of the insertion part 110. In addition, in order to prevent the central urethra 12b and the proximal urethra 12c from being irritated, an end portion surface of the insertion part 110 may be upwardly inclined with respect to the anteroposterior direction. Accordingly, a contact area between the apparatus 100 for treating urinary incontinence and the anterior vaginal wall is limited to a degree that can stop the incontinence, so that a wearer's foreign body sensation may be reduced when the apparatus 100 for treating urinary incontinence is worn.

Meanwhile, the locking part 120 includes an anterior extension part 121 that extends toward the anterior direction and a posterior extension part 122 that extends toward the posterior direction. According to an embodiment of the present disclosure, a length of the posterior extension part 122 may be longer than a length of the anterior extension part 121.

More specifically, when an end portion of the anterior extension part 121 is in contact with the urethra 12, the urethra 12 may be irritated and incontinence may be induced. Therefore, in order for the anterior extension part 121 to not contact the urethra 12, the anterior extension part 121 may extend such that the end portion of the anterior extension part 121 is positioned between the urethra 12 and the vagina 13. In this situation, by extending the posterior extension part 122 longer than the anterior extension part 121 such that the genital skin is caught on the posterior extension part 122, the insertion part 110 may be prevented from being inserted deeply into the vagina 13.

According to an embodiment of the present disclosure, a first extension length al1 that extends toward the anterior direction of the anterior extension part 121 may be at least 4 mm but less than or equal to 6 mm, and may preferably be about 5 mm. In addition, a second extension length al2 that extends toward the posterior direction of the posterior extension part 122 may be at least 16 mm but less than or equal to 24 mm, and may preferably be about 20 mm. However, the second extension length al2 is not limited to the value described above, and may extend such that the second extension length al2 has another value according to a degree of support of the insertion part 110 and to whether the wearer is uncomfortable.

Here, the first extension length all may refer to a length to the end portion of the anterior extension part 121 from a foremost point of the locking part 120 that is in contact with the insertion part 110. Further, the second extension length al2 may refer to a length to the end portion of the posterior extension part 122 from a rearmost point of the locking part 120 that is in contact with the insertion part 110.

FIG. 8 is a view schematically illustrating underpants provided with the apparatus for treating urinary incontinence according to an embodiment of the present disclosure.

According to an embodiment of the present disclosure, the apparatus 100 for treating urinary incontinence may be coupled to underpants 1000, and a wearer's convenience may be increased. More specifically, the underpants 1000 may include a wearing part 1100 formed to be worn on a woman's body and a coupling part 1200 formed so that the apparatus 100 for treating urinary incontinence is mounted.

According to an embodiment of the present disclosure, the coupling part 1200 is formed of at least two layers of cloths, and the apparatus 100 for treating urinary incontinence may be coupled to the coupling part 1200 by being fitted between the cloth layers. Here, in the coupling part 1200 on a center part of the cloth that is in contact with the skin of the wearer, a hole corresponding to a cross-section of the insertion part 110 of the apparatus 100 for treating urinary incontinence may be formed.

Since the apparatus 100 for treating urinary incontinence is provided on the underpants 1000, the underpants 1000 can be adjusted via an outerwear. Accordingly, even if the apparatus 100 for treating urinary incontinence is not in contact with the specific position, a position of the apparatus 100 for treating urinary incontinence can be easily adjusted to a correct position by adjusting the underpants 1000 via the outerwear. Therefore, the wearer's convenience may be increased.

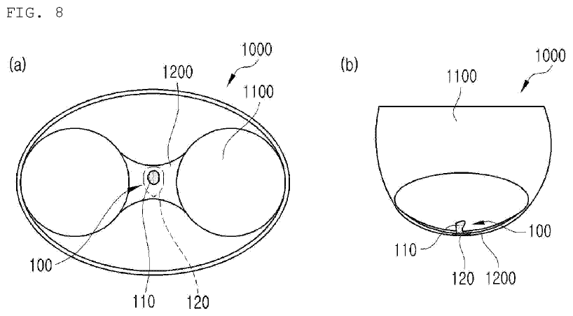

FIG. 7 is a plan view illustrating the apparatus for treating urinary incontinence according to an embodiment of the present disclosure.

According to another embodiment of the present disclosure, the locking part 120 may be formed such that a width thereof in a left and right direction is gradually decreased along the anteroposterior direction. That is, a width of the anterior extension part 121 may be smaller than a width of the posterior extension part 122. By this structure, a portion of the underpants is prevented from getting caught in the crevice between the buttocks, so that the wearer's wearing sensation may be improved.

While the present disclosure has been described above by way of particular features such as specific components and the like, and exemplary embodiments, these embodiments are provided to further facilitate overall understanding of the present disclosure, and the present disclosure is not limited thereto. Various modifications and variations may be made from the above descriptions by those skilled in the art.

Therefore, the spirit of the present disclosure should not be limited to the above-described embodiments, and not only the append claims but also all those modified equally or equivalently to the claims are intended to fall within the scope of the spirit of the present disclosure.

### DESCRIPTION OF REFERENCE NUMERALS

10: body

11: clitoris

12: urethra

13: vagina

13B: first point

14: anus

100: apparatus for treating urinary incontinence

110: insertion part

111: contact part

112: support part

110a: outer circumferential surface

120: locking part

120a: first surface

121: anterior extension part

122: posterior extension part

### INDUSTRIAL APPLICABILITY

The present disclosure is applicable to the field of an apparatus for treating urinary incontinence.

1. An apparatus for treating urinary incontinence, the apparatus comprising:
a locking part provided with a first surface that is in contact with a genital skin of a woman; and
an insertion part which protrudes from the first surface of the locking part and which extends to a predetermined length such that the insertion part is inserted into the woman's vagina,
wherein the insertion part comprises:
a contact part that protrudes forward from the insertion part such that the contact part is in contact with a first point on a front side of the woman's vaginal wall, the first point corresponding to a connection part between the woman's distal urethra and the woman's central urethra; and
a support part that protrudes rearward in an opposite direction from the contact part such that the support part is in contact with a rear side of the woman's vaginal wall,
wherein the support part protrudes longer than the contact part such that a state in which the contact part is in contact with the first point is maintained.

2. The apparatus of claim 1, wherein the insertion part is configured such that a cross-sectional area thereof is gradually decreased and then increased from at a part where the contact part or the support part is formed to the locking part.

3. The apparatus of claim 2, wherein the contact part protrudes by at least 2 mm but less than or equal to 4 mm from a foremost point that is spaced apart by a predetermined distance from the first surface of the locking part in a length direction of the insertion part.

4. The apparatus of claim 3, wherein the support part protrudes by at least 5 mm but less than or equal to 7 mm from a rearmost point that is spaced apart by the predetermined distance from the first surface of the locking part in the length direction of the insertion part.

5. The apparatus of claim 4, wherein the predetermined distance is at least 10 mm but less than or equal to 15 mm.

6. The apparatus of claim 5, wherein a foremost protruding point of the contact part is positioned closer to the first surface of the locking part than a rearmost protruding point of the support part.

7. The apparatus of claim 6, wherein the foremost protruding point is spaced apart from the first surface of the locking part by at least 20 mm and less than or equal to 25 mm.

8. The apparatus of claim 7, wherein an end portion surface of the insertion part is upwardly inclined with respect to a front-to-back direction.

9. The apparatus of claim 7, wherein a preset length of the insertion part is less than or equal to 35 mm.

10. The apparatus of claim 1, wherein the locking part comprises an anterior extension part that extends forward and a posterior extension part that extends rearward, and the posterior extension part extends longer than the anterior extension part.

11. The apparatus of claim 10, wherein the anterior extension part extends such that an end portion of the anterior extension portion at the front side is positioned between the woman's urethra and the woman's vagina in order for the anterior extension part to be not in contact with the woman's urethra when the insertion part is inserted into the woman's vagina.

12. The apparatus of claim 10, wherein a width of the posterior extension part in a left and right direction is gradually decreased toward a rear side of the posterior extension part.

13. Underpants comprising:
an apparatus for treating urinary incontinence according to claim 1;
a wearing part which forms an external shape of the underpants and which is formed to be worn on a woman's body; and
a coupling part formed such that the apparatus for treating urinary incontinence is mounted.

14. The underpants of claim 13, wherein the coupling part is formed of at least two layers of cloths, and a locking part is coupled to the underpants by being fitted between the cloth layers.
